(19) United States
(12) Patent Application Publication
(10) Pub. No.: US 2025/0221748 A1
(43) Pub. Date: Jul. 10, 2025

(54) SURGICAL PLAQUE

(71) Applicant: BEZMIALEM VAKIF UNIVERSITESI, Istanbul (TR)

(72) Inventor: Banu KILIC, Istanbul (TR)

(73) Assignee: BEZMIALEM VAKIF UNIVERSITESI, Istanbul (TR)

(21) Appl. No.: 18/854,996

(22) PCT Filed: Dec. 31, 2020
(86) PCT No.: PCT/TR2020/051481

§ 371 Date: Oct. 8, 2024

(30) Foreign Application Priority Data
Dec. 30, 2020 (TR) 2020/22430

(51) Int. Cl. A61B 17/80

(52) U.S. Cl. CPC A61B 17/8071

(57) ABSTRACT

At least one surgical plaque is provided, which has at least one fixation plaque so as to fix the separated bone parts of the lower and upper jaws in orthognathic surgery. Accordingly, its novelty is to include the following: at least one vertical extension that is connected from one side of said fixation plaque so as to fix the broken parts of 1^{st} mandibula, and at least one carrier element provided to move linearly on said vertical extension so as to provide skeletal anchorage.

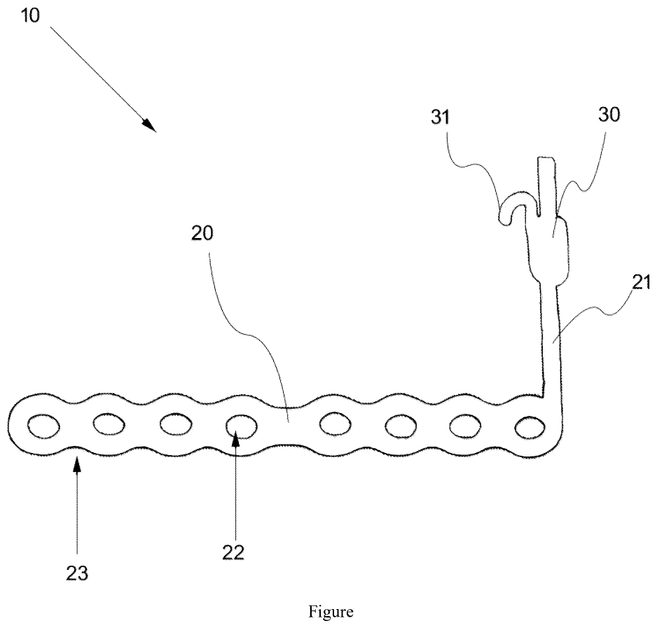

### CROSS REFERENCE TO THE RELATED APPLICATIONS

This application is the national phase entry of International Application No. PCT/TR2020/051481, filed on Dec. 31, 2020, which is based upon and claims priority to Turkish Patent Application No. 2020/22430, filed on Dec. 30, 2020, the entire contents of which are incorporated herein by reference.

### TECHNICAL FIELD

The present invention particularly relates to at least one surgical plaque having at least one fixation plaque so as to fix the separated bone parts of the lower and upper jaws in orthognathic surgery.

### BACKGROUND

Orthognathic surgery is a surgical method that helps to correct the deformities related to jaws. In traditional orthognathic surgery approach, the patient is prepared for surgery by making dental arrangements with orthodontic treatment prior to surgery, surgical operation is performed under general anesthesia, and ideal occlusion is provided with orthodontic treatment that is carried out after surgery. In the surgery first (SF) method that is popular in the recent years, the bones are placed in their ideal positions with the surgery first method and subsequently the teeth are aligned in these new positions of the bones. The popularity of this novel method is rapidly increasing since it has many advantages such as the patient's determining the time of surgery, accelerated orthodontic treatment, increasing the effectiveness of decompensation etc. However, a stable postoperative occlusion is impossible because orthodontic treatment is not carried out prior to the surgery in this method. This problem is solved with surgical splints carried after surgery and 4 extra plaques or 8 mini screws that are placed in the posterior during surgery for vertical control. Since the regions where the plaques are located allow limited anatomic opportunity, placing them with the stabilization plaques may be a problem. It leads to extra trauma and cost for the patient. In cases where skeletal anchorage systems are not used, as the vertical dimension increases, the position of the mandibula has become more unpredictable in postoperative treatment. Positioning the used plaques is closely associated with the required orthodontic movement and the force application point on the plaque varies according to the needs of the orthodontist.

There are plaques produced for the application of orthodontic force for the lower and upper jaws. Fixation plaques for the lower and upper jaw are also available and used. However, there is no such a plaque design that can provide fixation of surgically separated segments and also provide skeletal anchorage for the orthodontist.

As a result, all abovementioned problems have made it necessary to make an improvement in the relevant technical field.

### SUMMARY

The present invention is related to a surgical plaque in order to eliminate the abovementioned disadvantages and to bring new advantages to the relevant technical field.

An object of the invention is to provide a surgical plaque that functions as both a fixation plaque and an orthodontic anchor plaque.

The present invention is at least one surgical plaque having at least one fixation plaque so as to fix the separated bone parts of the lower and upper jaws in orthognathic surgery so as to fulfill all objects mentioned above and obtained from the following detailed description. Accordingly, its novelty is to comprise the following; at least one vertical extension that is connected from one side of said fixation plaque so as to fix the broken parts of mandibula,
at least one carrier element that is provided such that it can move linearly on said vertical extension so as to provide skeletal anchorage. Therefore, in the surgical process, the bones are fixed and then it is allowed to make modification in the desired position vertically by the orthodontist.

Another possible embodiment of the invention is characterized in that; it comprises at least one hook provided on at least one side of said carrier element. Therefore, it can be provided that the skeletal anchor is made in the desired position.

A possible embodiment of the invention is characterized in that; the vertical extension is a rectangular protrusion that is at least partially engaged to the fixation plaque at a right angle.

Therefore, the carrier element is provided to perform linear movement easily and it is made possible to apply force couple.

Another possible embodiment of the invention is characterized in that; said carrier element is essentially configured to be a tube with four corners and partially in the form of cylinder. Therefore, the carrier element can be modified according to different jaw structures and surgery.

Another possible embodiment of the invention is characterized in that; said carrier element is essentially configured to be a + shaped double tube. Therefore, the carrier element can be modified according to different jaw structures and surgery.

Another possible embodiment of the invention is characterized in that; said carrier element is essentially configured to be a bracket head tube. Therefore, the carrier element can be modified according to different jaw structures and surgery.

Another possible embodiment of the invention is characterized in that; said carrier element is essentially configured to be a perforated tube. Therefore, the carrier element can be modified according to different jaw structures and surgery.

Another possible embodiment of the invention is characterized in that; said carrier element is essentially configured to be a lip bumper tube. Therefore, the carrier element can be modified according to different jaw structures and surgery.

Another possible embodiment of the invention is characterized in that; said carrier element is essentially configured to be a slider insertion tube. Therefore, the carrier element can be modified according to different jaw structures and surgery.

### BRIEF DESCRIPTION OF THE DRAWINGS

An illustrative perspective view of the inventive surgical plaque is given in FIGURE.

### REFERENCE NUMBERS

10 Surgical plaque
20 Fixation plaque
21 Vertical extension
22 Hole
23 Formed section
30 Carrier element
31 Hook

### DETAILED DESCRIPTION OF THE EMBODIMENTS

In this detailed description, the inventive surgical plaque (10) is described by means of examples only for clarifying the subject matter such that no limiting effect is created.

An illustrative perspective view of the inventive surgical plaque (10) is given in FIGURE. Accordingly, said surgical plaque (10) is essentially a plaque which allows for using the same with vertical anchorage purposes and the force application point of which can be changed in the post-operative period by the orthodontist according to the individual, to be used in the solution of post-operative vertical direction orthodontic problems in the orthognathic surgery cases. The surgical plaque (10) comprises at least one fixation plaque (20). Said fixation plaque (20) are plaques that are produced for the purpose of applying orthodontic force so as to fix the separated bone parts of the lower and upper jaws. The fixation plaque (20) comprises at least one hole (22). Said hole (22) is opened on the fixation plaque (20) sequentially and is a space to provide a connection with a screw. The fixation plaque (20) comprises at least one formed section (23). Said formed section (23) is essentially a recess provided on the surface between the holes (22) of the fixation plaque (20).

The fixation plaque (20) comprises at least one vertical extension (21). Said vertical extension (21) is essentially a flat bar and is provided at one end of the fixation plaque (20) and is provided such that there is at least partially right angle between them. The vertical extension (21) can perform the function for fixing the broken parts of the mandibula. In a possible embodiment of the invention, the vertical extension (21) is mainly an angular and particularly a rectangular bar. Since the vertical extension (21) is angular, it is possible to use the same with designs that will allow the forces to be applied by the orthodontist to be a force couple.

The surgical plaque (10) comprises at least one carrier element (30). Said carrier element (30) is provided in a manner such that it can essentially move linearly on the vertical extension (21). The carrier element (30) is placed so as to provide skeletal anchorage after surgical operation. The carrier element (30) is ensured in a rectangular form and can be fixed and re-activated according to the user's demand. In a possible embodiment of the invention, the carrier element (30) is configured to be a hollow cylindrical tube, + shaped double tube, bracket head tube, perforated tube (22), lip bumper or slider insertion tube. Since the carrier element (30) is linearly movable in the vertical extension (21), it allows modification in different positions and sizes by the orthodontist after surgery, independent of the physical positioning of the same by the surgeon. The carrier element (30) is engaged with at least one hook (31). Said hook (31) allows determining the region where the force will be applied and performing the operation by placing the plaque.

In a representative embodiment of the invention, the fixation plaque (20) is located so as to fix the separated bone parts of the lower and upper jaws during orthognathic surgical operation. A carrier element (30) is positioned on the vertical extension (21) after the operation so as to provide skeletal anchorage for the orthodontist and to solve orthodontic problems in the vertical direction. The plaque is located in the desired position and force can be applied by moving the carrier element (30) linearly.

The orthodontic anchor plaque and a further fixation plaque (20) to be applied to the patient during surgery is not required with the use of surgical plaque (10) together with all this configuration and therefore the amount of anesthesia to be received by the patient will be reduced as the number of plaques is reduced by half. It reduces the amount and risk of complications that may occur due to the surgeon's concentration loss and fatigue because the duration is shortened. The amount of used screws is reduced with the use of a single surgical plaque (10), thus this reduces the risk of complications due to mini-screws. The positioning of the plaque is facilitated by calculating the position of the part to be removed from the mucosa during the fixation of the segments by the surgeon since it will not be necessary to place a vertical plaque. Furthermore, the carrier element (30) located on the vertical extension (21) of the surgical plaque can be selected and attached by the orthodontist in the post-surgical recovery period with different properties and its position can also be changed. This located carrier element (30) can be replaced with new ones later on in accordance with the needs of the orthodontist without damaging the vertical extension (21). In case the vertical extension (21) is not required, the vertical extension (21) of the fixation plaque (20) can be cut and removed with simple incisions under local anesthesia. The surgical plaque (10) and fixation plaques (20) placed during orthognathic surgery are used for post-operative orthodontic anchorage purposes.

The protection scope of the invention is specified in the appended claims and cannot be limited to the description made for illustrative purposes in this detailed description. Likewise, it is clear that a person skilled in the art can present similar embodiments in the light of the above descriptions without departing from the main theme of the invention.

What is claimed is:

1. A surgical plaque, comprising:
at least one fixation plaque configured to fix separated bone parts of lower and upper jaws in an orthognathic surgery,
at least one vertical extension, wherein the vertical extension is connected from a side of the fixation plaque to fix broken parts of a mandibula,
at least one carrier element provided to move linearly on the vertical extension to provide a skeletal anchorage, and
at least one hook provided on a side of the carrier element.

2. (canceled)

3. The surgical plaque according to claim 1, wherein the vertical extension is a rectangular protrusion, wherein the rectangular protrusion is at least partially engaged to the fixation plaque at a right angle.

4. The surgical plaque according to claim 1, wherein the carrier element is essentially configured to be a tube with four corners and partially in a form of a cylinder.

5. The surgical plaque according to claim 1, wherein the carrier element is essentially configured to be a + shaped double tube.

6. The surgical plaque according to claim 1, wherein the carrier element is essentially configured to be a bracket head tube.

7. The surgical plaque according to claim 1, wherein the carrier element is essentially configured to be a lip bumper tube.

8. The surgical plaque according to claim 1, wherein the carrier element is essentially configured to be a slider insertion tube.
